(19) United States
(12) Patent Application Publication
(10) Pub. No.: US 2013/0116547 A1
(43) Pub. Date: May 9, 2013

(54) Measurement of Anaplerotic Flux by Hyperpolarization Transfer

(71) Applicants: Matthew E. Merritt, Euless, TX (US); Craig R. Malloy, Dallas, TX (US)

(72) Inventors: Matthew E. Merritt, Euless, TX (US); Craig R. Malloy, Dallas, TX (US)

(73) Assignees: The United States Government as represented by the Department of Veteran Affairs, Washington, DC (US); The Board of Regents of the University of Texas System, Austin, TX (US)

(21) Appl. No.: 13/621,567

(22) Filed: Sep. 17, 2012

Related U.S. Application Data
(60) Provisional application No. 61/535,175, filed on Sep. 15, 2011.

(51) Int. Cl. A61B 5/055; A61B 5/00

(52) U.S. Cl. CPC A61B 5/055; A61B 5/0042; A61B 5/0044; A61B 5/004
USPC 600/420

(57) ABSTRACT

Methods and composition for metabolic imaging are provided. For example, in certain aspects, methods for hyperpolarization transfer combined with hyperpolarization are provided. Furthermore, the invention provides methods for detecting magnetic resonance signals for biological processes such as anaplerosis.

### CROSS-REFERENCE TO RELATED APPLICATIONS

This application claims priority to U.S. Provisional Application No. 61/535,175 filed Sep. 15, 2011, the entire contents of which is specifically incorporated herein by reference without disclaimer.

### STATEMENT REGARDING FEDERALLY SPONSORED RESEARCH

This invention was made with government support under 5P41-RR002584-23 awarded by the National Institute of Health. The government has certain rights in the invention.

### BACKGROUND OF THE INVENTION

1. Field of the Invention

The present invention relates generally to the field of nuclear magnetic resonance imaging. More particularly, it concerns measurement of biological processes, such as anaplerosis, by magnetic resonance.

2. Description of Related Art

Anaplerosis refers to the process of synthesizing new molecules such as glucose (by the liver and other tissues), neurotransmitters (by the brain and other tissues), and amino acids (by the kidney and other tissues). Anaplerosis may involve the utilization of molecules for biosynthetic purposes and for degrading molecules that occur during normal or abnormal cellular metabolism. The process of anaplerosis induces the citric acid cycle.

The citric acid cycle serves two major functions, energy production and biosynthesis. The oxidation of acetyl-CoA provides the vast majority of energy for almost all cells in the body. In this process, the addition of a two-carbon acetyl unit is balanced by the release of two carbon atoms as CO₂ in one turn of the cycle with no change in the concentration of intermediates of the cycle. Transitions from low to high workloads do not require any change in the concentration of citric acid cycle intermediates. The heart, for example, is capable of modulating citric acid cycle flux over a very wide range with no change in the concentration of intermediates. The citric acid cycle is also involved in the catabolism of amino acids and odd carbon fatty acids, and it serves a key role in numerous biosynthetic processes.

Anaplerosis is of physiological importance because all tissues with mitochondria must be able to replenish citric acid cycle intermediates as they are used for biosynthetic processes. There are at least three important anaplerotic mechanisms in mammalian tissue, all of which are essential to life. One critical pathway is the pyruvate carboxylase pathway which generates oxaloacetate from pyruvate and CO₂. This pathway is used for neurotransmitter synthesis from pyruvate in the brain, gluconeogenesis from amino acids in the liver, glyceroneogenesis from lactate (required for triglyceride synthesis), or catabolism of certain amino acids. Another important pathway is the group of reactions, including metabolism of odd-carbon fatty acids and some amino acids, that terminate in propionyl-CoA which is converted to succinyl-CoA in many tissues including the heart, liver, kidney and other tissues. The delivery of amino acids—glutamate, glutamine, aspartate—to the cycle is also a direct source of carbon skeletons. The magnitude of anaplerotic sequences depends on the tissue, the nutritional state and disease. In the liver, for example, pyruvate carboxylation is the key enzyme for gluconeogenesis and the addition of carbon skeletons is balanced by their removal via phospho-enol pyruvate carboxykinase. Hepatic gluconeogenesis from the citric acid cycle during fasting in humans is 300%-600% of citric acid cycle flux. Activity in other tissues is less, relative to the citric acid cycle, but for the most part very little information is available about these fluxes in vivo.

Anaplerosis is a basic feature of metabolism but the only general method for its measurement available until now requires ¹³C NMR using conventional technology. Because of low signal using conventional methods, it is difficult or impossible to apply in many experimental preparations or patients. Thus, there remains a need for methods involving detection of a wide range of biological processes, particularly anaplerosis, with high sensitivity magnetic resonance.

### SUMMARY OF THE INVENTION

Aspects of the present invention overcome a major deficiency in the art by providing novel methods for transfer of hyperpolarization in living subjects, tissues or organs. In a first embodiment, the method may comprise obtaining a living subject, organ, or tissue that comprises a hyperpolarized target, the hyperpolarized target comprising detectable isotopes such as isotopes with a nonzero nuclear spin at a first and second position. In certain aspects, the first position has a hyperpolarized isotope. For detection information on the second position with hyperpolarization sensitivity, the method may further comprise transferring the hyperpolarization from the first position to the second position. In certain aspects, the first position may have a longitudinal relaxation time (T₁) longer than that that of the second position. The first position may be a storage position for storing hyperpolarized nuclei, while the second position may be an informative position for providing information of a desirable biological process. The third position may also carry important information related to anaplerosis.

In further aspects, one or both of the isotopes are not ¹H. For example, the T₁ at the first position has a value of at least or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 seconds, or any intermediate ranges or numbers. For example, the T₂ at the second position has a value of at most or about 0.01, 0.05, 0.1, 0.2, 0.3, 0.4, 0.5, 0.6, 0.7, 0.8, 0.9, 1, 2, 3, 4, 5, 6, 7, 8, 9, 10 seconds, or any intermediate ranges or numbers. Non-limiting examples of the isotope at the first or second position include ¹³C, ¹⁹F, ³¹P, ³³S, ¹⁵N, ⁸⁹Y, ⁷⁵As, ⁶³Cu, ⁶⁵Cu, ²³Na, ⁹⁹Ru, ¹⁰¹Ru, ⁶Li, ⁷Li, ¹³³Cs, and ¹⁷O. In particular, the isotope is ¹³C or ¹⁵N.

The method may be used for measurement or detection of a number of biological processes. In certain aspects, the hyperpolarized target may be an intermediate in an in vivo biological process, such as anaplerosis, glucose metabolism, ornithine cycle, GABA (γ-Aminobutyric acid) cycle, beta oxidation, tricarboxylic (TCA) cycle or urea cycle. In a particular aspect, this invention could be used to elucidate the presence of “anaplerotic flux” in living systems. For example, the hyperpolarized target may be hyperpolarized glutamate for imaging a process involving citric acid cycle, such as anaplerosis.

For obtaining the subject, tissue or organ, the method may comprise introducing into the subject, tissue or organ a hyperpolarized precursor which may provide the hyperpolarized target to be detected in the subject, tissue or organ. For example, the introducing method may be injection, more particularly intravenous injection. The hyperpolarized precursor may be hyperpolarized by any hyperpolarization methods such as dynamic nuclear polarization (DNP), para-hydrogen induced polarization (PHIP), or brute force polarization. For metabolic imaging, the hyperpolarized precursor may be a hyperpolarized metabolite or an intermediate in a metabolic process, for example, a tricarboxylic acid (TCA) cycle metabolite, such as a hyperpolarized form of [U-¹³C]pyruvate, [2,3-¹³C]pyruvate, [1,2-¹³C]acetate, [U-¹³C]lactate, [U-¹³C]alanine, [1,2-¹³C]acetyl-CoA, [U-¹³C]butyrate, [1,2-¹³C]butyrate, or any compound that could produce [1,2-¹³C]acetyl-CoA.

To enrich detectable isotopes at the position(s) to be detected in the target, especially the informative positions, the method may comprise administering to the subject, tissue or organ a precursor labeled with an isotope of the same type as that of the isotope at the second position of the hyperpolarized target, such as by infusion or perfusion. In a particular aspect, the isotope to be enriched is ¹³C. For example, the labeled precursor is [U-¹³C]pyruvate, [U-¹³C]acetate, [U-¹³C]propionate, [1-¹³C]pyruvate, [3-¹³C]pyruvate, [U-¹³C]lactate, [U-¹³C]alanine, [U-¹³C]dihydroxyacetone, [1-¹³C]lactate, [1-¹³C]alanine, [3-¹³C]lactate, or [3-¹³C]alanine. Preferably, the isotope enrichment may be prior to or during the step of introduction of hyperpolarized precursors.

The hyperpolarized precursor, as described above, may be administered into a tissue, an organ or a subject, for example by injection. In certain aspects, the first position has a relatively long T₁ and thus may preserve the hyperpolarization at a desirable time scale. After the hyperpolarized precursor is introduced into the desired tissue, organ or subject, a polarization transfer method may be used to transfer hyperpolarization from the first position to the second position which is to be detected. The polarization transfer may be homonuclear transfer, such as C—C transfer, or heteronuclear transfer between any two different nuclei. Any methods known in the art may be used for such polarization transfer, like a J-coupling scheme, such as the use of FLOPSY (Flip-flop spectroscopy), MLEV (Malcolm Levitt's composite-pulse decoupling sequence), DIPSI (Decoupling in the presence of scalar interactions), WALTZ, or HOHAHA (Homonuclear Hartmann-Hahn), or a J-modulated scheme, such as the use of INADEQUATE (Incredible natural abundance double quantum transfer) or DOUBTFUL (Double quantum transitions for finding unresolved lines), or any selective pulse versions of these transfer schemes.

For example, inside the MRI system, an MR “pulse sequence” for polarization transfer may be used to transfer the hyperpolarization from the C5 position of glutamate to the C4 or C3 position in the subject, tissue or organ. Thus, enrichment of detectable isotopes at the C4 or C3 position, for example as developed by the infusion of ¹³C precursors, may be read out by MR with high sensitivity due to the transfer of hyperpolarization from the C5 position to the C3 position.

In a further aspect, the method may comprise transferring the hyperpolarization from the first position to an intermediate position before transferring to the second position, or the second position may comprise at least two distinct positions. For example, the hyperpolarization may be transferred from the C5 position to the C4 position and then from the C4 position to the C3 position in the glutamate.

For imaging of a biochemical process in the subject, tissue or organ, the method may comprise detecting magnetic resonance signals in the target after the hyperpolarization transfer. The method may further comprise measuring such a biochemical process in the subject, tissue, or organ based on the magnetic resonance signals. Non-limiting examples of the biological process may be anaplerosis, glucose metabolism, ornithine cycle, GABA (γ-Aminobutyric acid) cycle, beta oxidation, urea cycle, or TCA cycle. Based on the measurement as compared to a control, the method may further comprise providing a prognosis or diagnosis of the subject.

The subject may be determined to have or be at risk of having a tumor, an inflammation, an infection, a metabolic disease, a neurological disease, a cardiac disease, a liver disease, a kidney disease, or diabetes. The subject may be a mammal, such as a human or mouse. The organ may be heart, brain, liver, or kidney, such as an ischemic or malignant organ or any tissue or organ having mitochondria. The tissue may be heart, brain, liver, or kidney tissue.

Embodiments discussed in the context of methods and/or compositions of the invention may be employed with respect to any other method or composition described herein. Thus, an embodiment pertaining to one method or composition may be applied to other methods and compositions of the invention as well.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Enhancement of protonated butyrate carbons following FLOPSY-8 mixing. The ¹³C NMR spectra of hyperpolarized [U-¹³C]butyrate after a 30° excitation pulse (bottom) was acquired two seconds prior to the FLOPSY-8 enhanced spectrum (top). The top spectrum shows the impact of 13 cycles of evolution, followed by the same 30° excitation pulse. The absolute phase is not preserved due to the non-zero transverse components of the average Hamiltonian that describes the FLOPSY-8 sequence. In addition, the C3 resonance exhibits a change in phase for the central peaks of the multi-component resonance.

FIG. 2: Absolute (top) and relative (bottom) enhancements of C2, C3, and C4 of [U-¹³C]butyrate as a function of FLOPSY-8 cycles. A total of n=4 enhancements were used to produce the error bars. *P<0.0001; **P<0.05 (except for C3 values).

FIG. 3: ¹³C NMR spectrum of hyperpolarized [U-¹³C]glutamate. The bottom panel shows the signal after an initial 30 degree excitation pulse. The top panel shows the spectrum after a 13 cycle FLOPSY-8 mixing period prior to a 30 degree excitation pulse, 2 seconds after the bottom spectrum was collected.

FIG. 4: Detection of ¹³C Enrichment in oxaloacetate after polarization transfer. Many ¹³C-enriched precursors will be metabolized by tissues to generate ¹³C-labeled oxaloacetate (gray circles; C3 and C4). Introduction of hyperpolarized [2,3-¹³C]pyruvate or [1,2-¹³C]acetate results in the production of [1, 2-13C]acetyl-CoA (dark circles), which can condense with pre-labeled oxaloacetate to make various isotopomers of citrate and glutamate. However, the C2 position of acetyl-CoA is protonated, and hence the polarization will decay rapidly (faded circles; C4 of glutamate). Once the label appears in the pool of the high concentration metabolite glutamate, FLOPSY-8 can be used to transfer polarization to the C4 and C3 positions, allowing a high sensitivity readout of oxaloacetate enrichment, and hence anaplerosis.

FIG. 5: Diagram of labeling patters derived from different isotopomers of acetyl-CoA condensing with an oxaloacetate labeled during a previous turn of the TCA cycle. Choice of labeling in TCA cycle precursors can be used to make estimates of substrate preference.

FIG. 6: The spectrum recorded using FLOPSY-8 and hyperpolarization as compared to standard thermally polarized 13C NMR is different due to the polarization pathway. Note that when a ¹³C nucleus is present only at the C3 position, no resonance is observed following FLOPSY-8 transfer since the C5 is not labeled (row 3). This loss of information must be accounted for in the metabolic modeling.

FIG. 7: Unlocalized ¹³C in vivo spectrum of a mouse following injection of [1-¹³C]pyruvate using the homebuilt polarizer #2 operating at 1.15 K. The extra resonances marked have not been assigned.

FIG. 8: Initial 3T implementation of ¹³C EPSI with 1.6 kHz BW and 5-mm resolution.

FIG. 9: Reconstruction using a conventional ¹H CSI (top) and a twice accelerated SENSE-CSI (bottom). Note the congruency of the two images and the spectra.

### DESCRIPTION OF ILLUSTRATIVE EMBODIMENTS

To increase the sensitivity of imaging of biological processes such as anaplerosis in living systems, methods are provided for transferring hyperpolarization from a long T₁ storage nucleus at the first position to one or more distal short T₁ nuclei at the second position, thereby allowing detection of these nuclei with high sensitivity long after their initial polarized states have decayed. Non-limiting examples of the nuclei at either positions include ¹³C, ¹⁹F, ³¹P, ³³S, ¹⁵N, ⁸⁹Y, ⁷⁵As, ⁶³Cu, ⁶⁵Cu, ²³Na, ⁹⁹Ru, ¹⁰¹Ru, ⁶Li, ⁷Li, ¹³³Cs, and ¹⁷O, preferably ¹³C-enriched carbon(s). The method may use precursor compounds labeled in one, two, three, or more sites for enrichment of labeled targets.

In certain aspects, method provided herein may allow detection of protonated nuclei such as carbons late after the initial dissolution, which could substantially expand the range of metabolic pathways that can be probed using hyperpolarized biological precursors. One value of this method is that it enables, for the first time, detection and potentially imaging of key biological pathways such as anaplerosis in vivo. It is contemplated, for example, that images of the rate of neurotransmitter synthesis could be generated.

In a particular embodiment, the method may be used to measure anaplerosis in a living system. For example, such a method may include four steps:
1) Administration, such as perfusion or infusion, of stable isotope (e.g., ¹³C)-labeled compounds may be used to enrich the pool of the target metabolite to a sufficient level in a desired organ or animal. For example, perfusion of a target organ or intravenous infusion with a ¹³C labeled precursor may be used to induce labeling in the intermediates that compose the Kreb's cycle.
2) A hyperpolarized precursor that, when metabolized, produces [1,2-¹³C]acetyl-CoA may be used. For example, this can be done by using [U-¹³C]pyruvate, [2,3-¹³C]pyruvate, or [1,2-¹³C]acetate. The compound, hyperpolarized using methods such as the DNP process, may be administered into the desired organ or animal.
3) Once the hyperpolarized imaging agent is injected inside a MRI system, an MR “pulse sequence” for polarization transfer may be used to transfer the polarization from the C5 position of glutamate to the C3 position. Enrichment of the C3 position, as developed by the administration of ¹³C precursors in step 1, may be read out by MR.
4) The enrichment levels of the C3 position can be modeled mathematically to produce estimates of anaplerotic flux in vivo. To the knowledge of the inventors, such measurements have not previously been made in vivo.

### I. ISOTOPE ENRICHMENT

In a certain embodiment, ¹³C labeled compounds may be used to enrich the pool of a labeled target metabolite, such as oxaloacetate (OAA), to a sufficient level. For example, a suitable ¹³C-enriched precursor such as [2-¹³C]acetate or other short chain fatty acid may be administered, such as by intravenous infusion, and metabolized in the citric acid cycle or via anaplerosis. The rate of citric acid cycle flux or anaplerotic flux may be calculated by monitoring the ratio of appearance of ¹³C in specific carbon positions of glutamate with signals enhanced by hyperpolarization transfer, and fitting to developed mathematical equations that give analytical solutions while obviating the need for modeling of the differential equations.

For example, living target tissues may be supplied with labeled precursors, such as [U-¹³C]pyruvate, until substantially steady-state conditions are achieved. At that point the distribution of ¹³C in glutamate, particularly at informative positions, may reflect metabolic fluxes into the citric acid cycle, including anaplerotic flux. Such methods may be utilized in the step of isotope enrichment by perfusion.

However, since the ¹³C is thermally polarized sensitivity may be poor. After substantial steady-state is achieved under thermally-polarized conditions, the corresponding hyperpolarized precursor, such as [U-¹³C]pyruvate, may be introduced. Hyperpolarized [1,2-¹³C]acetyl CoA may enter the citric acid cycle. Because of T₁ decay, glutamate C4 may quickly lose its longitudinal magnetization. At this point the ¹³C labeling in carbon 3 originates from the prior period of perfusion and may be thermally polarized. The ¹³C labeling in carbon 4 originates from the hyperpolarized material, but it may have returned to equilibrium. Hyperpolarization could be retained on glutamate C5. Polarization transfer methods such as FLOPSY-8 may be used to distribute longitudinal magnetization from C5 to C4 to C3. The hyperpolarized ¹³C signal from C4 and C3 may “read out” the isotopomers established in the isotope enrichment step prior to administration of hyperpolarized ¹³C.

In certain aspects, this technical advance in magnetic resonance (broadband polarization transfer) paired with the methods for measuring metabolic information based on isotopomer analysis could allow physiologically important parameters like substrate selection and TCA cycle turnover to be assayed with high sensitivity. In addition, this new method may allow these physiological parameters to be estimated without to the need for modeling the time course of hyperpolarized magnetization

### II. HYPERPOLARIZATION

It is anticipated that any, or essentially any, hyperpolarization methods known in the art may be used to obtain hyperpolarized precursors. The hyperpolarized precursors may be administered to a living system to provide target metabolites with hyperpolarized positions, e.g., for storage of hyperpolarization in a high T₁ nucleus.

Hyperpolarization of precursors, such as ¹³C-nuclei-containing molecules, may be achieved by different methods including, e.g., those described in WO-A-98/30918, WO-A-99/24080 and WO-A-99/35508, which are incorporated herein by reference, and hyperpolarization methods including polarization transfer from a noble gas, “brute force”, spin refrigeration, the parahydrogen method, and dynamic nuclear polarization (DNP).

Non-limiting examples of hyperpolarized precursors include [U-¹³C]pyruvate, [2,3-¹³C]pyruvate, [1,2-¹³C]acetate, [U-¹³C]lactate, [U-¹³C]alanine, [1,2-¹³C]acetyl-CoA, [U-¹³C]butyrate, [1,2-¹³C]butyrate, and any compound that can produce [1,2-¹³C]acetyl-CoA. For example, to obtain hyperpolarized pyruvate, one may polarize the pyruvate directly or polarize ¹³C-pyruvic acid and convert the polarized ¹³C-pyruvic acid to polarized ¹³C-pyruvate, e.g. by neutralization with a base.

One suitable way for obtaining a hyperpolarized precursor is the polarization transfer from a hyperpolarized noble gas, e.g., as described in WO-A-98/30918. Noble gases having non-zero nuclear spin can be hyperpolarized by the use of circularly polarized light. A hyperpolarized noble gas, preferably He or Xe, or a mixture of such gases, may be used to effect hyperpolarization of ¹³C-nuclei. The hyperpolarized gas may be in the gas phase, it may be dissolved in a liquid/solvent, or the hyperpolarized gas itself may serve as a solvent. Alternatively, the gas may be condensed onto a cooled solid surface and used in this form, or allowed to sublimate.

Another suitable way for obtaining hyperpolarized precursor involves imparting polarization to ¹³C-nuclei by thermodynamic equilibration at a very low temperature and high magnetic field. Hyperpolarization compared to the operating field and temperature of the NMR spectrometer is effected by use of a very high field and very low temperature (brute force). Higher magnetic field strength is generally preferable, e.g., higher than 1 T, preferably higher than 5 T, more preferably 15 T or more, and even more preferably 20 T or more. Lower temperatures are generally preferable, e.g. 4.2 K or less, preferably 1.5 K or less, more preferably 1.0 K or less, even more preferably 100 mK or less.

Another suitable way for obtaining hyperpolarized precursor is the spin refrigeration method. This method covers spin polarization of a solid compound or system by spin refrigeration polarization. Generally, the system is doped with or intimately mixed with suitable crystalline paramagnetic materials such as Ni²⁺, lanthanide or actinide ions with a symmetry axis of order three or more. The instrumentation is typically simpler than required for DNP, with no need for a uniform magnetic field since no resonance excitation field is applied. The process is carried out by physically rotating the sample around an axis perpendicular to the direction of the magnetic field. The pre-requisite for this method is that the paramagnetic species has a highly anisotropic g-factor. As a result of the sample rotation, the electron paramagnetic resonance may be brought in contact with the nuclear spins, leading to a decrease in the nuclear spin temperature. Sample rotation is carried out until the nuclear spin polarization has reached a new equilibrium.

In a particular embodiment, DNP (dynamic nuclear polarization) is used to obtain a hyperpolarized precursor. In DNP, polarization of MR active nuclei in a compound to be polarized is affected by a polarization agent or so-called DNP agent, a compound comprising unpaired electrons. During the DNP process, energy, normally in the form of microwave radiation, is provided, which will initially excite the DNP agent. Upon decay to the ground state, there is a transfer of polarization from the unpaired electron of the DNP agent to the NMR active nuclei of the compound to be polarized, e.g., to the ¹³C nuclei in ¹³C-pyruvate. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarization enhancement is achieved may be employed. The DNP technique is further described, e.g., in WO-A-98/58272 and in WO-A-01/96895, both of which are incorporated by reference herein.

To polarize a compound by the DNP method, a mixture of the compound to be polarized and a DNP agent is prepared (“a sample”) that is either frozen and inserted as a solid into a DNP polarizer for polarization or is inserted into a DNP polarizer as a liquid and freezes inside said polarizer due to the very low surrounding temperature. After the polarization, the frozen solid hyperpolarized sample may be rapidly transferred into the liquid state either by melting it or by dissolving it in a suitable dissolution medium. Dissolution is preferred. A dissolution process for producing a frozen hyperpolarized sample, and suitable devices therefor, are described in detail in WO-A-02/37132. The melting process and suitable devices for the melting are described, e.g., in WO-A-02/36005.

In order to obtain a high polarization level in the compound to be polarized said compound and the DNP agent generally need to be in intimate contact during the DNP process. This is not the case if the sample crystallizes upon being frozen or cooled. To avoid crystallization, glass formers may be included in the sample or compounds may be chosen for polarization that do not crystallize upon being frozen but rather form a glass.

### III. HYPERPOLARIZATION TRANSFER

To increase detection sensitivity of polarization of specific positions, polarization transfer methods may be employed. Incorporation of polarization transfer can allow estimates of biological processes such as metabolic flux and substrate selection using the sensitivity of hyperpolarization without being limited to modeling of differential equations. Polarization transfer methods such as FLOPSY may be performed to readout or obtain the metabolic data encoded in informative positions, such as in carbons 4 and 3 of glutamate.

Depolarization of nuclei due to the inherent longitudinal relaxation is one of the inherent challenges of using hyperpolarized nuclei, and this is especially true for protonated carbons which often have a T₁ in the ˜2 second range. However, if a suitable nucleus with a long T₁ is nearby and can serve to store hyperpolarization for an extended period, a method may be provided for the detection of the shorter-lived (short T₁), but highly informative, nuclei. The method may transfer polarization from the long T₁ nucleus via polarization transfer methods such as coherent mixing schemes to “re-polarize” the target short T₁ nuclei. The schemes may transfer polarization between the two sets of nuclei while preserving information about isotope enrichments. Thus, the chances of observing protonated carbons at informative positions in living tissues may be enhanced.

Any polarization transfer methods that are known in the art may be employed, including WALTZ (Shaka et al., 1983), DIPSI (Decoupling in the presence of scalar interactions) (Rucker and Shaka, 1989), FLOPSY-8 (FLip-flOP SpectroscopY) (Mohebbi and Shaka, 1991), MLEV (Malcolm Levitt's composite-pulse decoupling sequence), and HOHAHA (Homonuclear Hartmann-Hahn). All of the references are incorporated herein in their entirety. As the field strength of NMR magnets has increased, the need for mixing schemes with broader excitation bandwidths (BW's) has also increased, hence the proliferation of pulse sequences. In a particular example, FLOPSY-8 may be used.

FLOPSY-8 (FLip-flOP SpectroscopY) is broad-band, and the bandwidth of the r.f. pulses impacts its success. FLOPSY-8 could be used to distribute longitudinal magnetization from C5 to C4 to C3. The hyperpolarized ¹³C signal from C4 and C3 could “read out” the isotopomers established prior to administration of hyperpolarized ¹³C. In a particular aspect, FLOPSY-8 may work properly on the 10 mm ¹³C probe where a mouse heart could be studied. FLOPSY-8 can be developed on two instruments, the 600 MHz with a 10 mm cold probe where the mouse heart can be studied, and on the 4.7T where r.f. bandwidth issues are easier to handle.

FLOPSY-8 polarization transfer was first introduced as a broadband homonuclear recoupling method for liquid state NMR in 1990 (Kadkhodie et al., 1990). Recent modifications using adiabatic pulses have further improved performance (Bennett et al., 2003). FLOPSY is now part of standard pulse sequence libraries on most NMR spectrometers. FLOPSY in transfer of hyperpolarized carbon-13 may need multiple mixing cycles to achieve the transfer. Since T₁ and T₂ decay is active during the sequence, it was not known if polarization transfer could be performed in an acceptably short time period. Polarization transfer from a long T₁ site through multiple short T₁ carbons has been achieved in Examples described below. A new algorithm for making in vivo estimates of anaplerosis may be contemplated using uniformly labeled hyperpolarized precursors of acetyl-CoA.

In particular, FLOPSY-8 is a computer optimized scheme that is known to have superior bandwidth coverage. It is based upon supercycling of a base sequence R, which is composed of a series of 9 pulses of various flip angles and phases, R=46₀ 96₄₅ 164_{67.5} 159₃₁₅ 130_{22.5} 159₃₁₅ 164_{67.5} 96₄₅ 46₀ where the subscript denotes the phase. The overall supercycle is R R̅R̅R RR R̅R̅, where the overbar implies a 180° overall phase shift. The sequence functions by causing a rotation in the zero-quantum subspace of two coupled spins, resulting in efficient exchange between the |+−> and |−+> levels. This flip-flop transition provides the name of the sequence, FLip-flOP SpectroscopY. (Kadkhodie et al., 1990). It is part of the Varian pulse sequence library and is easily incorporated into new pulse sequences.

### IV. METHODS OF DIAGNOSIS

In further aspects, the invention may be used to detect or provide a diagnosis or prognosis of a disease characterized by generation of new molecules at an abnormal rate, such as type 2 diabetes, cancer, or various mental illnesses. For example, type 2 diabetes is thought to be a consequence of excess hepatic glucose production. In this instance, the unregulated production of glucose drives up the concentration of glucose in the blood and a frequently used drug, metformin, is thought to act by inhibiting glucose production. Drugs may be selected to target excess glucose production so that it could aid therapy for type 2 diabetes. Thus, the method may be used to detect biological processes, such as anaplerosis, in a desired living subject, tissue or organ in the presence or absence of a test drug.

Cancer cells generate new molecules to grow and divide. It is anticipated that excess anaplerosis may be diagnostically characteristic of certain cancers. In various embodiments, methods of the present invention may be used to identify abnormal metabolic rates in the cancer patients and use the information for cancer diagnosis.

Various mental illnesses and psychological disorders can arise from abnormalities in neurotransmitter synthesis. Methods of the present invention may be used, in various embodiments, to detect neurotransmitters, such as GABA, glutamate, and/or glutamine, that are generated by the citric acid cycle via anaplerotic pathways. Thus, in various aspects, the methods of the present invention may be used to detect or measure the altered synthesis of at least one neurotransmitter associated a mental illness or psychological disorder. Identification of altered neurotransmitter synthesis in a subject may be helpful for diagnosing and/or treating the subject for a psychological disorder or mental illness.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Transfer of Hyperpolarization from Long T₁ Storage Nuclei to Short T₁ Neighbors Using FLOPSY-8

Samples of [U-¹³C]butyrate and [U-¹³C]glutamate were polarized in an Oxford HyperSense DNP system (Oxford Instruments Molecular Biotools, Tubney Woods, UK) using a water/glycerol or water/DMSO glassing matrix and 15 mM concentration of the trityl radical (Oxford Instruments).

A standard solution of 5.5 M sodium [U-¹³C]butyrate in de-ionized (DI) H₂O was prepared by neutralization of [U-¹³C]butyric acid (Sigma-Aldich, Isotec) with 10 M NaOH (aq). For DNP, trityl radical (Tris(8-carboxyl-2,2,6,6-tetra[2-(1-hydroxyethyl)]-benzo(1,2-d:4,5-d′)bis(1,3)dithiole-4-yl)methyl sodium salt) (Oxford Instruments Molecular Biotools Ltd, Oxfordshire, UK) was added to a final concentration of 15 mM. The samples were placed in the 3.35 T Oxford HyperSense ((Oxford Instruments Molecular Biotools Ltd, Oxfordshire, UK)) DNP system and polarized for ˜1.25 hours. The electron irradiation frequency was set to the positive lobe of the DNP enhancement curve (94.072 GHz). The sample was held at 1.4 K during the polarization. Samples were dissolved by injecting 4 mL of boiling DI H₂O. Three different protocols were used to assess the effect of delays in delivering the sample from the HyperSense to the detection magnet. For absolute enhancement measurements (protocol 1), the entire volume was transferred in 8 seconds directly to a 10 mm NMR probe positioned in a VNMRS 400 MHz (89 mm bore Oxford magnet) spectrometer (Agilent Instruments, Santa Clara, Calif.). To examine the effects of collecting the sample in the stray field of the magnet prior to injection (protocol 2), the sample was dissolved and deposited in a beaker near the bore of the spectrometer, where 0.8 mL was transferred to a 5 mm outer diameter NMR tube, then placed in the spectrometer immediately. To allow for the near total destruction of the polarization of the protonated carbons (protocol 3), the sample was allowed to stand for an additional 15 seconds at high field (center of the magnet) prior to the first detection pulse.

After transfer, a 30 degree excitation pulse was applied to the ¹³C channel for detection without proton decoupling to eliminate possible confounding enhancements from NOE effects as compared to DNP. Two seconds after the first detection pulse, the FLOPSY-8 sequence was initiated for a variable number of cycles, followed by detection with another 30 degree ¹³C excitation pulse. The RF field strength during the FLOPSY mixing was 13.8 kHz. Enhancements were determined by fitting spectra in magnitude mode using ACD (Advanced Chemistry Development, Toronto, Canada).

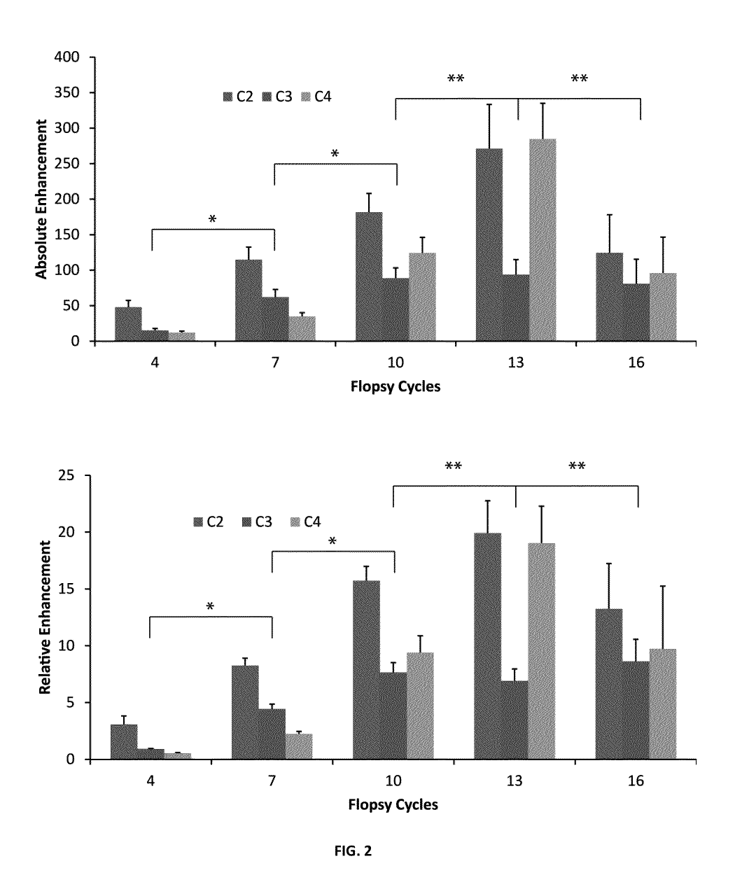

A standard solution of 100 mM sodium [U-¹³C]glutamate in (1:1) DI H₂O:Glycerol was prepared by neutralizing [U-¹³C]glutamic acid (Cambridge Isotope Laboratories) with concentrated NaOH (aq). An equal volume of glycerol was added to the aqueous sodium [U-¹³C]glutamate solution to attain the final standard solution. For DNP, trityl radical was added to a final concentration of 15 mM. The samples were placed in the 3.35 T Oxford HyperSense DNP system and polarized for ˜6 hours. The electron irradiation frequency was set to the positive lobe of the DNP enhancement curve (94.072 GHz). The sample was held at 1.4 K during the polarization. Protocol 3 was followed after samples were dissolved by injecting 4 mL of boiling DI H₂O. FLOPSY data was acquired similarly to the [U-¹³C]butyrate samples. All experiments were repeated 4 times (n=4). Spectra were processed using ACD NMR processor (Toronto, Canada) in phased mode for the enhancements in protocol 1 and 2, and in the magnitude mode for protocol 3. Data are presented as mean±standard deviation and two-tailed t-tests were used to assess differences for FIG. 2. All statistical analyses were performed with 95% confidence level using GraphPad Prism version 5.03 (GraphPad Prism Software, Inc, La Jolla, Calif.).

Absolute enhancements for the [U-¹³C]butyrate were ˜3500 above the thermal polarization when measured with a minimum transfer time of 8 seconds (protocol 1). For experiments where the sample was loaded into a 5 mm NMR tube inside the fringe field of the magnet and then placed in the magnet, enhancements were ˜30% lower (Table 1) for the carboxyl carbon and even lower for the protonated carbons. Adding an additional ˜15 s delay (protocol 3) resulted in an enhancement of only ˜600 for the carboxyl carbon. It was at this time point that the FLOPSY-8 sequence was initiated.

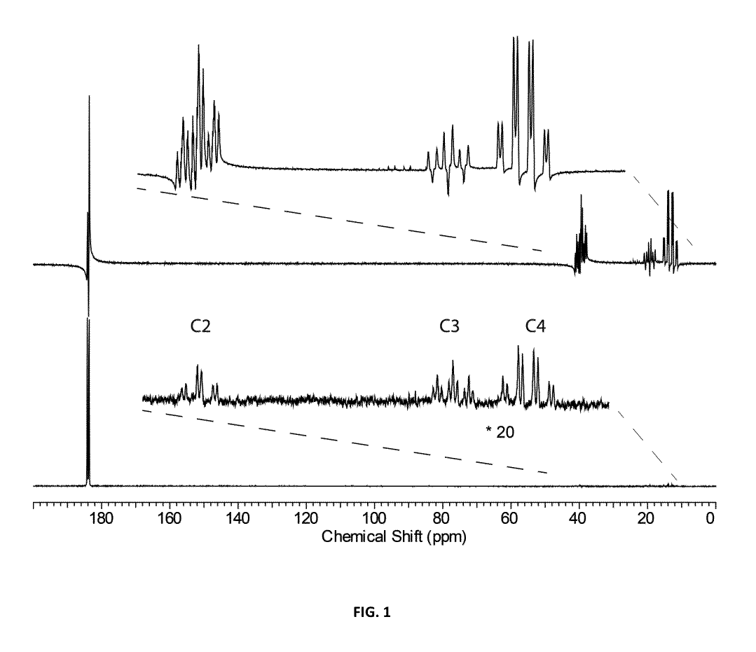

The effect of FLOPSY-8 on the ¹³C NMR spectrum of hyperpolarized [U-¹³C]butyrate is illustrated in FIG. 1. The bottom spectrum shows the protonated carbons of butyrate after the first 30 degree pulse. The ¹J_{CC} for C1 to C2 is 51 Hz, while the ¹J_{CC} between the C2 and C3 and C3 and C4 is degenerate, being ˜35 Hz in both cases. Therefore, the apparent triplet of triplets for the C3 resonance is due to a ¹J_{CH} coupling as well as coupling to its two carbon neighbors, resulting in overlapping resonances associated with the central peaks of the doublet of doublets arising from the uniform ¹³C labeling. In the top spectrum (FIG. 1), polarization was transferred from the C1 to the C2, C3, and C4 carbons in a total mixing time of ˜22 ms (13 FLOPSY-8 cycles). FLOPSY-8 is a longitudinal mixing sequence; therefore an excitation pulse is still required after the mixing to excite transverse magnetization. Since butyrate has only a single long T₁ nucleus, this result demonstrates that this experimental protocol can transfer polarization along a chain of at least three protonated ¹³C-enriched carbons.

TABLE 1
Calculated Spin-Lattice Relaxation and Pre-FLOPSY
Enhancement of Sodium [U-¹³C₄]Butyrate.

| | |
|---|---|
| Butyrate | Pre-FLOPSY Enhancement^{b} |

| Carbon | T₁ (sec)^{a} | 1 sec delay | 25 sec delay |
|---|---|---|---|
| 1 | 30 | 4000 | 2500 |
| 2 | 6 | 1200 | —^{c} |
| 3 | 6 | 1200 | —^{c} |
| 4 | 6 | 1200 | —^{c} |

^{a}Determined using standard inversion-recovery methods at 37° C.
^{b}Determined after a single 32° excitation pulse and comparing the peak area to a thermally acquired peak area with a 90° excitation pulse.
^{c}Signal not detected.

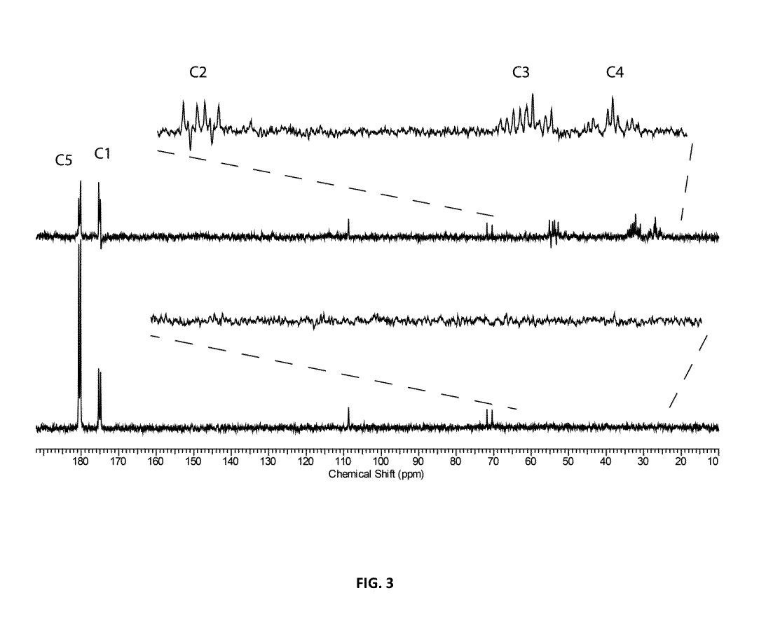

The choice of mixing time for optimal polarization transfer was explored empirically (FIG. 2). Mohebbi, et. al., showed that for the two spins of [U-¹³C]acetate at 75 MHz detection frequency, the optimal mixing time consists of a total of 10 cycles of FLOPSY-8 evolution. For our case (100 MHz ¹³C frequency) longer mixing times (13 cycles) were needed for optimal transfer. FIG. 2 plots both the absolute enhancement (top) of the protonated carbons as compared to the thermal NMR signal versus the total number of cycles as well as the relative enhancements (bottom) as measured compared to the initial 30 degree pulse on the hyperpolarized sample. The absolute and relative enhancements were significantly different for the C2 and C4 carbons at each choice of number of mixing cycles. The C3 was significantly lower for 4 and 7 cycles, but an evolution period of 10, 13, or 16 cycles did not produce a significant difference in C3 enhancement. Table 1 includes both the T₁s of the butyrate carbons as well as the absolute enhancements of each prior to the beginning of the mixing sequence. As can be seen, FLOPSY-8 restores the absolute enhancement of the C2 and C4 carbons to a level that is about half that of the carbonyl carbon. The measured C3 carbon enhancement is lower, primarily due to the anomalous phase of the central peak associated with the overlapping doublet of doublets. This appears to be a phenomenon associated with the hyperpolarization of the signals, as FLOPSY-8 experiments using a thermally polarized sample of [U-¹³C]butyrate did not show the out of phase character typically seen here (see supplementary material).

[U-¹³C]glutamate differs from butyrate in that it has two long T₁ carboxylic acid groups at the C1 and C5 positions of the molecule, both with T₁s ˜14 seconds (add ref here, Badar-Goffer et al., 1990). The spectrum following the initial 30° excitation shows both the C1 and C5 carbons as well as the glycerol from the glassing matrix (FIG. 3). Residual magnetization from the protonated carbons of glutamate was not observed using this relatively small flip angle. Thirteen cycles of FLOPSY-8 mixing results in the spectrum shown in the top of FIG. 3, which displays some of the anomalous intensities for the C2 carbon at 55 ppm as seen for the C3 carbon of [U-¹³C]butyrate. In this case ¹J_{CC}¹² (54 Hz) and ¹J_{CC}²³ (35 Hz) are not degenerate. The relative and absolute enhancements for C2, C3 and C4 were 75, 54, and 76 times the thermal spectrum. Due to the short T₁ of the protonated glutamate carbons, the bottom spectrum of FIG. 3 represents the thermal spectrum of these resonances; these carbons have returned to thermal polarization prior to the FLOPSY-8 mixing.

FLOPSY-8 is effective for transferring polarization from long-T₁ storage nuclei to other J-coupled carbons in both [U-¹³C]butyrate and [U-¹³C]glutamate. The presence of two long T₁ carbons in glutamate does not apparently provide any benefit in increasing the polarization of the protonated carbons. However, the condition of having both the C1 and C5 carbons of glutamate in a highly polarized state could not exist in vivo. As demonstrated by Tyler, et. al., hyperpolarized [5-¹³C]glutamate produced from [2-¹³C]pyruvate can be detected in the perfused rat heart (Schroeder et al., 2009). This observation indicates that considerable polarization is maintained in at least four downstream metabolites (citrate, isocitrate, α-ketoglutarate and glutamate) as hyperpolarized [2-¹³C]pyruvate is metabolized in the TCA cycle. Glutamate is produced in vivo following the condensation of acetyl-CoA with oxaloacetate, with the two carbons of acetyl-CoA becoming the C4 and C5 carbons of glutamate. The C1 position of glutamate can only be labeled after multiple turns of the TCA cycle, so it is highly probable that all hyperpolarization of the spins would have decayed back to thermal equilibrium by the time the enrichment could arrive in C1. If FLOPSY-8 were to be used for in vivo or in vitro measurements of Kreb's cycle kinetics, the transfer would therefore come from the C5 position alone. For this reason, we chose to use the [U-¹³C]butyrate as a mimic of in vivo production of glutamate.

Optimization of the mixing time for butyrate empirically produced a maximum at 13 cycles of mixing; this differs with the original FLOPSY-8 papers which restricted the mixing to a two-spin model system (Mohebbi and Shaka, 1991). However, FLOPSY-8 mixing has a marked dependence on the shift between the resonances observed, as evidenced in references 12 and 14. As confirmation, the same experiment shown here was duplicated for [U-¹³C]acetate at 4.7 T. With half the B₀ field strength, only 7 cycles of FLOPSY-8 mixing resulted in maximum transfer between the coupled spins (data not shown). Increasing efficiency of mixing at lower frequencies means that carrying out this experiment in vivo at 4.7 T, 3 T, or even 1.5 T should become markedly easier to accomplish, requiring progressively less B₁ amplitude. For the experiments shown here at 100 MHz ¹³C frequency, other TOCSY type mixing sequences such as DIPSI-2 (Rucker and Shaka, 1989) and MLEV-16 (Levitt et al., 1981) performed much worse than FLOPSY-8 in producing polarization transfer. The adiabatic version of the sequence (Bennett et al., 2003) was not attempted for these experiments as currently it is not easily generated in the Agilent software. It is predicted that moving to lower detection frequencies would allow a variety of mixing sequences to become effective in transferring the polarization. Sequential transfer schemes using shaped pulses would also be a viable option provided that the T₂ of the detected carbons was not a limiting factor.

For [U-¹³C]butyrate, an absolute enhancement of ˜300 was observed for the C2 carbon adjacent to the long T₁ C1 carbon, which itself began with a polarization of ˜600 prior to the mixing period (Table 1). FLOPSY-8 can produce higher polarization transfer values for isolated two-spin systems, however, this is not the case for samples which are uniformly labeled in ¹³C. For [U-¹³C]butyrate, the transfer achieved here appears in line with that which could be achieved for a thermally polarized sample. New experiments that include proton decoupling will explore the effects of the proton J-coupling and NOE upon the absolute enhancements. It is not clear why the polarization transfer drops so precipitously at 13 cycles of mixing (FIG. 2). While an answer to this might be of substantial theoretical interest, the results of mixing for 13 cycles were sufficient to allow us to develop the ideas outlined below.

The polarization transfer results suggest a method for making a fundamentally new in vivo measurement of metabolic function using uniformly labeled pyruvate or acetate. In the case where the glutamate in a tissue was pre-labeled using an infusion of ¹³C labeled substrates prior to the HP injection, isotopomers would be formed by the condensation of the [1,2-¹³C]acetyl-CoA with ¹³C labeled oxaloacetate. FLOPSY-8 transfer from the C5 to C3 position, via the ¹³C labeled C4 position, would provide a direct readout of C3 enrichment, and the J-coupled multiplets there could be used to measure anaplerotic flux into the Krebs cycle via known methods (Sherry et al., 2004). While the enhancements measured here are an order of magnitude lower than that typically achieved with hyperpolarized [1-¹³C]pyruvate, that is not necessarily an obstacle to performing the experiment suggested above in cell culture or in vivo. The measurement of anaplerosis depends upon relative areas of the peaks in the multiplets, not upon measuring a time course of magnetization evolution; the measurement only needs a single spectrum. With only this precondition, a single 90 degree pulse that consumes all of the magnetization (and therefore provides maximum sensitivity) would be necessary for the measurement. In addition, the enhancements achieved here for butyrate are much less than that achievable with pyruvic acid or acetate. Future experiments implementing this scheme with either of these precursors of acetyl-CoA would likely start with polarizations 3 or 4 times as high as those shown here for butyrate. With the proper modeling of the polarization transfer through the entire spin system using a program like SPINACH (Hogben et al., 2011), then the protocol for measuring anaplerosis suggested above does not need enhancements greater than those shown here.

Without wishing to be bound by any theory, FIG. 4 shows a scheme by which the interior carbons of citrate and glutamate could be assayed using a hyperpolarized, uniformly labeled precursor of acetyl-CoA paired with FLOPSY-8 polarization transfer. Anaplerosis is the import of carbon skeletons into the Kreb's citric acid cycle for essential biosynthetic processes such as generation of amino acids that serve as neurotransmitters in the brain or glucose synthesis in the liver during fasting. Pre-labeling of a tissue such as the heart or liver using a ¹³C labeled precursor results in enrichment in various positions of oxaloacetate. This information is not accessible by standard HP schemes, as the T₁s are too short to allow polarization to persist longer than 1 turn of the cycle. Polarization of the C5 position of glutamate is known to be observable following injection of [2-¹³C]pyruvate. In the case where the glutamate in a tissue was pre-labeled using an infusion of ¹³C labeled precursors prior to the injection of hyperpolarized precursors, isotopomers would be formed by the condensation of the hyperpolarized [1,2-¹³C]acetyl-CoA with ¹³C labeled oxaloacetate. FLOPSY-8 transfer of hyperpolarization signals from the C5 to C3 position of glutamate, via the ¹³C labeled C4 position, would provide a direct readout of C3 enrichment, and the J-coupled multiplets there could be used to measure anaplerotic flux into the Krebs cycle via known methods (Sherry et al., 2004). Using this method, hyperpolarized signals from the positions of glutamate derived from oxaloacetate (C3) should be detectable in vivo. Measurement of relative multiplet areas will then allow estimates of the contribution of anaplerosis as a fraction of total Kreb's cycle turnover.

In conclusion, a method for transferring polarization from hyperpolarized, long T₁ nuclei to J-coupled neighbors has been demonstrated. This method may be used to measure metabolism or anaplerosis in vivo.

### Example 2

### Imaging Anaplerosis with Hyperpolarized ¹³C

Reaction pathways feeding carbon into the citric acid cycle for biosynthetic purposes are termed “anaplerotic sequences”. These reactions play a central role in key synthetic processes yet there is no general method for specifically imaging these pathways. Hyperpolarized [U-¹³C]acetic acid and hyperpolarized [U-¹³C]pyruvic acid will be used to label carbons 4 and 5 of glutamate in the isolated heart. Since the effects of anaplerosis on the ¹³C spectrum of glutamate are due to changes in ¹³C enrichment in carbons 3, 2 and 1 of glutamate, flip-flop spectroscopy (FLOPSY-8) will probe ¹³C multiplets in protonated carbons of glutamate and measure in a few seconds the activity of anaplerotic reactions. The method can be validated with conventional isotopomer methods.

Part of the current method lies in the application of hyperpolarization transfer methods such as FLOPSY-8 to isolated tissue or living subjects and the integration of FLOPSY-8 data with NMR isotopomer analysis. The attraction of FLOPSY-8 is the ability to transfer hyperpolarization to protonated carbons where metabolic information can be “read out” with hyperpolarization sensitivity. The hyperpolarized carbon can “read” an adjacent, thermally-polarized ¹³C. Therefore, one can advantageously interrogate previously-established ¹³C labeling with hyperpolarization sensitivity. One can supply tissues or a patient with ¹³C-enriched precursors for some period of time, depending on the physiology and the clinical question. Once the desirable distribution of thermally polarized ¹³C is achieved, one can acquire this information with hyperpolarization sensitivity.

The ¹³C in position 4 and position 5 of glutamate provide the same information since both carbons are correlated and derive from acetyl-CoA. Detection of enrichment in carbon 4 by FLOPSY provides substantially no additional information, as compared to detecting carbon 5. However, carbons 1, 2 and 3 of glutamate are derived from oxaloacetate and, therefore, contain information about anaplerosis. The ability to detect the multiplets in C4 provides information about label in carbon 3 of glutamate, equivalent to carbon 2 of oxaloacetate. Similarly, detecting the multiplets in carbon 3 provides information about label in position 2 of glutamate (identical to carbon 3 of oxaloacetate). Consequently, FLOPSY-8 enhanced spectroscopy provides the potential to probe a class of reactions not previously investigated by hyperpolarization methods.

Tissues can be supplied with either [U-¹³C]acetate or [U-¹³C]pyruvate until substantial steady-state conditions are achieved. At that point the distribution of ¹³C in glutamate will reflect metabolic fluxes into the citric acid cycle. However, since the ¹³C is thermally polarized sensitivity will be poor. After substantial steady-state is achieved under thermally-polarized conditions, the corresponding hyperpolarized precursor, either [U-¹³C]acetate or [U-¹³C]pyruvate, can be introduced. Hyperpolarized [1,2-¹³C]acetyl CoA will enter the citric acid cycle. Because of T1 decay, glutamate C4 will quickly lose its longitudinal magnetization. At this point the ¹³C labeling in carbon 3 originates from the prior period of perfusion and is thermally polarized. The ¹³C labeling in carbon 4 originates from the hyperpolarized material, but it has returned to equilibrium. Hyperpolarization will be retained on glutamate C5. FLOPSY-8 can be used to distribute longitudinal magnetization from C5 to C4 to C3. The hyperpolarized ¹³C signal from C4 and C3 will “read out” the isotopomers established prior to administration of hyperpolarized ¹³C. Studies will use isolated rat hearts, isolated mouse hearts and the rat in vivo.

FLOPSY-8 is essentially broad-band, which means that bandwidth of the r.f. pulses impacts its success. FLOPSY-8 may not work on the 600 MHz a rat heart because of the diameter of the transmit coil. FLOPSY-8 can be developed on two instruments, the 600 MHz with a 10 mm cold probe where the mouse heart can be studied, and on the 4.7T where r.f. bandwidth issues are easier to handle.

The isolated Langendorf-perfused mouse heart can be studied in the 10 mm cold probe, and/or the rat heart can be studied in the 4.7 T system. The perfusate can be a modified Krebs-Henseleit (KH) medium maintained at 37° C. Polyethylene tubing attached to a pressure transducer will be inserted in the left ventricle across the mitral valve to measure left ventricular developed pressure (LVDP) and heart rate (HR) throughout the experiment. Myocardial O₂ consumption (MVO₂) can be calculated from the difference in O₂ tension between the perfusion medium in the arterial supply line and the coronary effluent. The heart can be supplied with [U-¹³C]pyruvate or [U-¹³C]acetate to substantially or essentially steady-state. Graded concentrations of propionate can be used to activate anaplerotic mechanisms. (Propionate is avidly metabolized in the heart via propionyl-CoA carboxylase that provides flux through succinyl-CoA into the citric acid cycle.) After steady-state is substantially achieved, HP [1,2-¹³C]acetate or HP [U-¹³C]pyruvate can be injected. FLOPSY spectroscopy can be used to detect the C3 and C4 of glutamate. Tissue can be freeze-clamped for conventional ¹³C NMR isotopomer analysis to directly measure anaplerosis and the labeling pattern of acetyl-CoA. Once the 4.7 T is available for ¹³C imaging, similar studies can be performed in the rat in vivo.

These results can be compared to the analysis tailored to FLOPSY data. FLOPSY-enhanced hyperpolarized ¹³C spectroscopy presents a particular challenge in isotopomer analysis. FLOPSY redistributes longitudinal magnetization from the hyperpolarized carbonyl of glutamate C5 to the C4 and C3 where ¹³C enrichment is detected by ordinary plus and acquire. The motivation for performing FLOPSY is to readout the metabolic data encoded in carbons 4 and 3 of glutamate. The principles of isotopomer analysis have been described in detail. If glutamate (with 32 isotopomers) is to be observed, then a 32×32 matrix can be written that describes the relative concentration of every one of the 32 glutamate isotopomers in terms of the biological variables of interest, in this case fc3 (doubly-labeled acetyl-CoA) and y (anaplerosis). If the concentration of all glutamate isotopomers is normalized to 1, then the concentration of [1,2,3,4,5-¹³C]glutamate is:

((fc3*fc3*(1−fc3)*(1+y+fc3))/((1+y)*(2*y+1)*(2+2*y)))*(fc3/(1−fc3))

For example, if 60% of acetyl-CoA is labeled in positions 1 and 2 (fc3=0.6), and if anaplerotic flux is 20% of citric acid cycle flux (y=0.2), then the percent of glutamate that is [1,2,3,4,5-¹³C]glutamate is 9.65%. Equivalent expressions can be derived for each of the other 31 glutamate isotopomers. As noted earlier, the ¹³C NMR spectrum of glutamate observes groups of isotopomers, and the mathematical expressions describing the NMR spectrum are substantially simpler than the expressions for individual isotopomers. Without wishing to be bound by any theory, these expressions derived many years ago were never published, possibly due to the lack of a general method for detecting individual isotopomers of glutamate or possibly due to the complexity of the expressions for each isotopomer, even under simple metabolic conditions.

The advent of hyperpolarization and FLOPSY made this information relevant. The equations can prove quite useful because only some of the FLOPSY-enhanced spectra correspond to the previously published NMR multiplet equations. The reason is that some isotopomers of glutamate such as [1,2,3-¹³C]glutamate will be detected in a thermally polarized spectrum with sufficient scan time, but cannot be detected during a FLOPSY experiment, since there is no “source” of hyperpolarized ¹³C in position 5. Only those isotopomers with label in carbons 5 and 4 and 3 can be detected in at ˜27 ppm, the chemical shift of carbon 3. FLOPSY detects only some of the glutamate isotopomers. Since the individual expressions relating each glutamate isotopomer to biological variables are available, one can derive the relation between the FLOPSY-enhanced spectrum and the metabolic variables. For example, under simple metabolic conditions (e.g., some fraction of the acetyl-CoA is [1,2-¹³C]acetyl-CoA, and there is flow of unlabeled carbon into the citric acid cycle via anaplerotic reactions), the triplet in carbon 3 due to J₂₃ and J₃₄ relative to the total signal in C3 is simply fc3/(y+1) in a FLOPSY-enhanced spectrum. In a thermally-polarized spectrum, this relation is different, fc3*fc3/(y+1).

### Example 3

### Methods for Measuring Absolute Flux Using Hyperpolarization

Methods for measuring physiological parameters that do not depend upon fitting of the hyperpolarization time vs. intensity curves would be valuable in studying a variety of disease states. Methods have been developed to pair pre-labeling with stable isotope precursors and hyperpolarization to produce estimates of turnover based on analytical models. In this Example, longitudinal mixing experiments are used to re-polarize protonated carbons to achieve this purpose. Longitudinal mixing of hyperpolarization can increase the likelihood of observing protonated carbons in living tissues. The short T₁'s of these carbons has previously made observation particularly difficult.

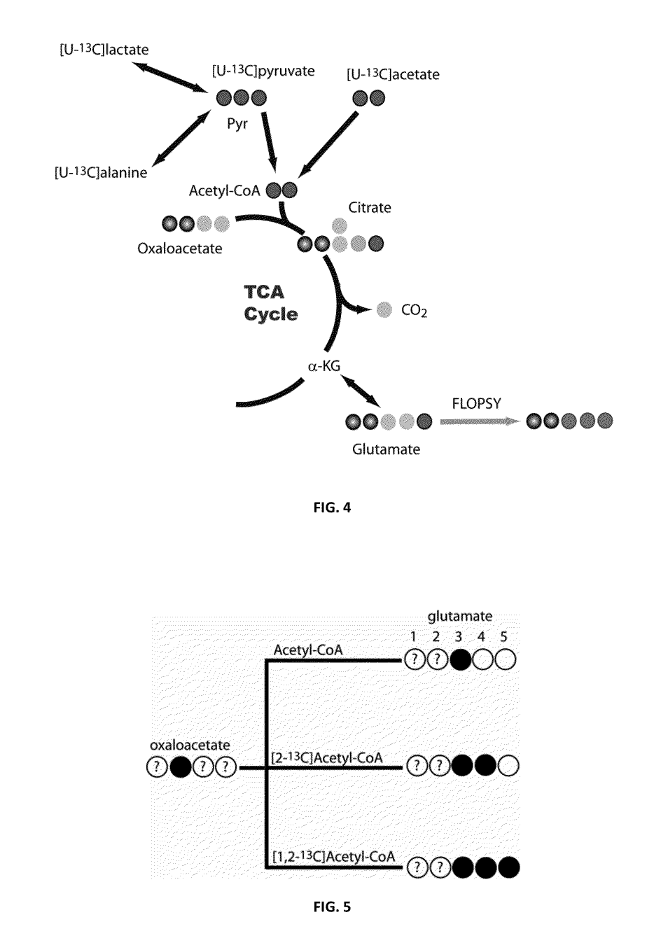

One parameter that is known to be influenced by the metabolic condition is substrate selection. Previously, it has been shown that in myocardial infarction followed by reperfusion, the rabbit heart showed a preference for acetate as compared to lactate or endogenous sources of nutrition such as glucose (Malloy et al., 1990a). This measurement was made under non-steady state conditions using a mixture of exogenous precursors including glucose, [U-¹³C]acetate, and [3-¹³C]lactate. A set of equations that do not rely upon achieving isotopic steady state in the heart and is generally applicable to all organs was used. FIG. 5 illustrates the scheme for making a substrate selection measurement that depends only upon the relative ratios of C3 to C4 and the relative contribution of the C4D34 coupling as compared to the rest of the C4 resonance in glutamate. The question marks in the diagram indicate possible labeling sites that are not relevant to making the estimate of substrate selection. A method using hyperpolarization that could duplicate this measurement (with the inherent sensitivity gain) would be a powerful tool for monitoring metabolism in living tissues.

One may make estimates using a tcaCALC type analysis (Malloy et al., 1990b) which depends on measuring the C3/C4 ratio as well as the relative multiplet areas of C2, C3, and C4. Analysis of this complexity has been completely outside the domain of hyperpolarization due to the fast T₁ decay of polarization associated with protonated carbons.

Longitudinal mixing of hyperpolarization from a long T₁ source to the aliphatic carbons of glutamate can be used for such experiments. FIG. 3 is a stack plot of two spectra taken of a sample of hyperpolarized [U-¹³C]glutamate. The lower spectrum was taken 8 seconds after dissolution in the HyperSense. The only resonances visible were from the C1 and C5 of glutamate and from the glycerol used in the polarization matrix. The upper spectrum was acquired 10 seconds after dissolution, but following a 15 ms mixing time using a FLOPSY-8 longitudinal mixing sequence to transfer polarization from the carboxyl groups of the glutamate to the protonated carbons. This result indicates that transfer of hyperpolarized magnetization works substantially the same as it does for thermal polarization in 2D and 3D protein structure elucidation experiments where FLOPSY-8 was intended for use. Further experiments where the C1 was depolarized with presaturation before FLOPSY-8 showed that polarization could be transferred from the C5 all the way to the C2, with the relative intensity C4>C3>C2. The equal relative intensities of FIG. 3 were derived from transfer from both of the highly polarized carbonyl carbons. Unequal intensities that occur in living tissues can be corrected by calibration experiments.

These results support the use of a new algorithm for taking advantage of hyperpolarization. First, a nucleus with sufficiently long T₁ to deliver polarization to the downstream metabolite of choice must be present. In certain embodiments, the 2-label of pyruvate may be used. Second, an infusion of ¹³C labeled compounds can be used to enrich the pool of the target metabolite to a sufficient level. And finally, a bolus of hyperpolarized material is added and the longitudinal mixing pulse sequence is used to transfer the polarization to the target nuclei already present due to the pre-labeling infusion. This technical advance in magnetic resonance (broadband polarization transfer) paired with the methods for measuring metabolic information based on isotopomer analysis will allow physiologically important parameters like substrate selection and TCA cycle turnover to be assayed with high sensitivity. In addition, these methods allow for physiological parameters to be estimated without having to resort to modeling the time course of hyperpolarized magnetization.

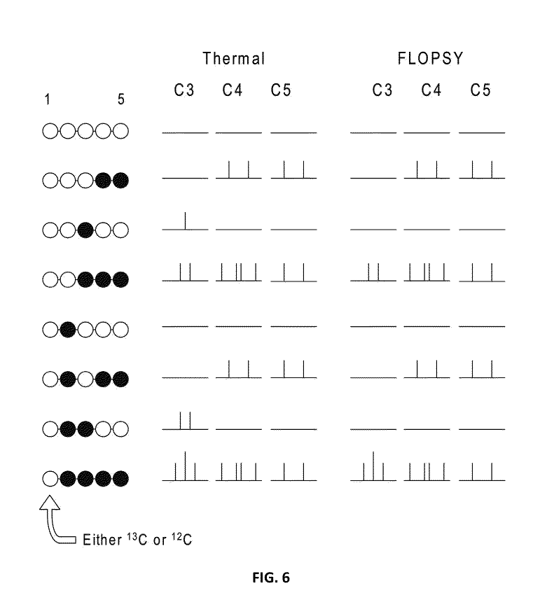

The recorded spectra can be different in the case of FLOPSY-8 transfer versus what would be recorded in a standard thermal ¹³C spectrum. FIG. 6 shows in diagram form the isotopomers that can be generated using hyperpolarized [U-¹³C]pyruvate paired with any sort of pre-perfusion experiment that produces labeling at the C3 of glutamate. Note that glutamate molecules that were not formed by condensation with [1,2-¹³C]acetyl-CoA will not appear in the FLOPSY-8 enhanced spectrum. This diagram assumes that polarization will proceed only down to the C3 carbon from the C5 position. C2 can also be polarized. The simplicity of the spectra can be described by a model of metabolism that can estimate anaplerosis in the TCA cycle, a parameter that, to the knowledge of the inventors, has heretofore has been outside the capabilities of hyperpolarization experiments.

### Example 4

### Adapt Current Chemical Shift Imaging (CSI) Technologies to the Detection of Hyperpolarized Nuclei at 4.7 T

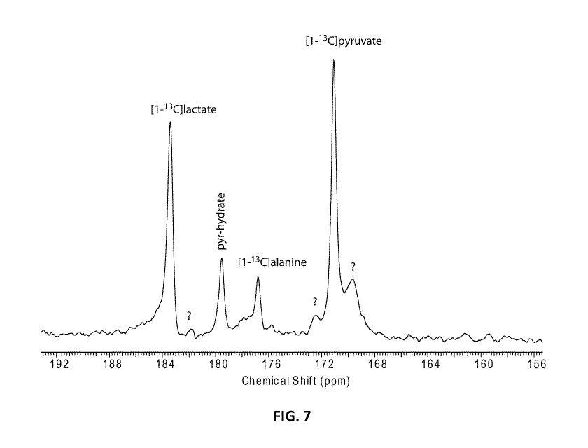

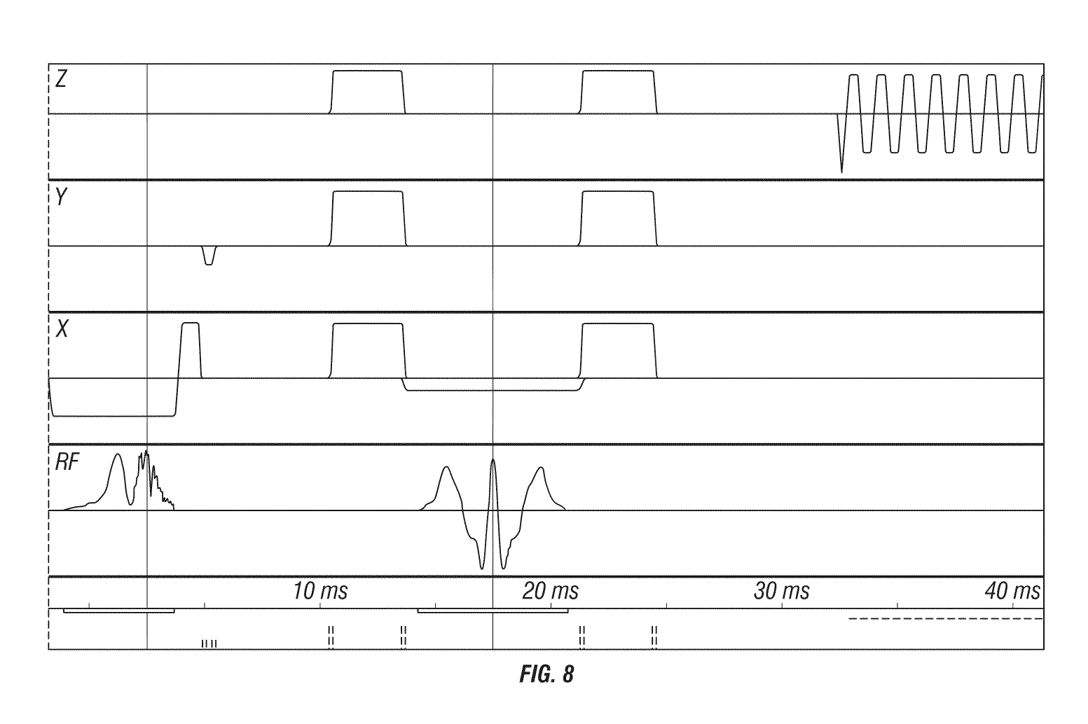

The homebuilt 4.6 T polarizer operating at 129 GHz electron frequency was used. The first in vivo results were obtained with a 300 μl injection of 80 mM [1-¹³C]pyruvate and small flip angle detection pulse (FIG. 7). The data was acquired using the 9.4 T vertical bore system. The third polarizer next to the 4.7 T horizontal bore system can be installed. This data illustrates that a working prepolarizer system is available for imaging experiments.

Adaptation of CSI protocols from 3 T to 4.7 T can be used for in vivo analysis. Cell culture, the perfused heart, and the perfused liver can be evaluated and/or used as models for studying metabolism. Alternately, whole animal studies can be performed.

Example 3 involves measuring enhancement in the C3 and C4 of glutamate alone, the resonances of which are separated by only 7 ppm. Thus, EPSI methods can be used. While the transfer of polarization can come for the C5 carbon of glutamate, the signal from this carbon is not necessary for estimates of metabolic parameters, and as such it is not necessary to have this resonance inside the detection bandwidth of the EPSI. Aliasing of the signal into the detection window can be performed, as a correct choice of offset can prevent overlap with the resonances of interest.

Another factor that may demand the application of EPSI methods in this case is that the FLOPSY-8 polarization transfer can depolarize the long T₁ polarization reservoirs (the carbonyls) almost completely. To take a second image, a new polarized agent can be perfused into the region of interest. Thus, a single shot CSI method can be advantageously used. Alternatively, the data acquisition can be restricted to single voxel spectroscopy methods, which might be superior for the small organ sizes seen in mice. INEPT transfer to the protons for detection uses 90 degree pulses, so the proton imaging can advantageously be executed as quickly as possible, or in the single voxel spectroscopy mode.

Establish Best CSI Protocols for ¹³C Detection at 4.7 T.

The bandwidth needed for detection of the C2 of pyruvate (˜205 ppm) to the CO₂ (˜125 ppm) is 4000 Hz. If the C3 of glutamate (˜27 ppm) is included in the needed bandwidth then the spectral width increases to 10,200 Hz. Therefore, ¹³C detection of metabolism would likely be most effectively carried out using the shallow flip angle 2D-CSI sequence like Golman and co-workers have shown. Running in a minimum Tᵣ mode (˜90 ms Tᵣ) they acquired over a 16*16 k-space using centric under-sampling to reduce the number of phase encodes to 149 in a total time of 13 seconds. The simplicity of this approach indicates that results can be immediately obtained on the Varian/Agilent systems without the coding/debugging of a new sequence. In addition, this method easily allows the incorporation of the wide spectral widths needed to record all the resonances one might expect in the full ¹³C spectrum. The 2D-CSI sequence can be adapted to the variable flip angle excitation schemes for signal excitation (Yen et al., 2009; Zeng et al., 2009; Zhao et al., 1996), which can reduce artifacts in the image derived from the decaying hyperpolarized magnetization as the phase encode steps are iterated through. Therefore, 2D-CSI will be the standard protocol used for validation of the 4.6 T turn-key polarizer paired with the 4.7 T imaging system.

With 2D-CSI established as a baseline for in vivo results, the flyback spin-echo CSI sequence of Cunningham can be coded and debugged for use at 4.7 T. This system is equipped with a set of fast switching gradients that can produce a maximum of 0.4 T/m gradient strength. A quick estimate of the bandwidth for ¹³C using this gradient set is:

$$BW = 10.638 \frac{MHz}{T} \times .4 \frac{T}{m} \times .06 m ≅ 255 kHz ,$$

or a “spatial” dwell time of about 4 μs assuming the 6 cm FOV. However, the maximum spatial bandwidth does not necessarily produce the optimal spectral bandwidth since the rewinding of the gradient back across k-space becomes a significant portion of the spectroscopic dwell time. A smaller maximum gradient strength can be weighted with faster rewinding to produce a larger spectral bandwidth. Using 8 spectral samples and 224 μs per echo, a bandwidth of 2237 Hz (or 44.7 ppm 13C frequency at 4.7 T) can be achievable for this sequence. This can cover the shifts of bicarbonate to [2-¹³C]pyruvate (205 to 161 ppm) or the C3 to C2 of glutamate (27-55 ppm). The total time of the readout and rewinding can be on the order of 450 μs, so 256 echoes can be acquired in ˜128 ms, which should be sufficient resolution for the spectra to resolve the j-couplings in each multiplet. Also, the total acquisition time should be within T₂* limits for the gradient echo readout. The flyback scheme has been successfully used to collect CSI images in as little as 3 seconds (Chen et al., 2007). This sequence can be juxtaposed against faster spiral CSI sequences, which require complicated reconstruction algorithms (Levin et al., 2007). The flyback double spin echo sequence appears to be a robust, fast chemical shift imaging sequence, and can be used as a target of pulse sequence development.

The simple single voxel techniques can be used to collect data in the organs of interest. The double spin-echo of PRESS would be the best sequence for in vivo spectroscopy of hyperpolarized magnetization, as it would have the same properties as the Cunningham sequence, i.e. the ability to use small flip angles for excitation while using the two 180 degree pulses in such a way as to leave the majority of the magnetization along the Z-axis (B₀).

Develop Schemes to Take Advantage of Polarization Transfer Imaging.

While both the FLOPSY-8 scheme and the INEPT scheme move polarization from one nucleus to the other, they do so in distinctly different ways. The FLOPSY-8 causes a longitudinal polarization transfer under an average Hamiltonian created by the windowless, multiple pulse sequence. Conversely, INEPT accomplishes the transfer in the transverse plane, and therefore the resulting magnetization must be handled in a different way than that created by FLOPSY-8.

The FLOPSY-8 is the easier of the two cases, since the <I_{z}> polarization generated by the transfer is the same as that present following the standard DNP experiments. Therefore, small flip angle schemes will work well, taking into account that the carbons that will be monitored will be protonated and can be expected to have T₁'s in the 1-2s range in vivo. As mentioned above, the bandwidth for detection of the protonated carbons of glutamate is limited to ˜30 ppm, which is small enough that the flyback spin echo method should work well.

The INEPT transfer to protons will have the benefit of detecting the very small frequency space of the lactate and alanine protons, or 30 Hz. With a bandwidth this small, very fast imaging using the flyback sequence would be possible. The key is restoring the generated magnetization to the z-axis of the rotating frame prior to executing the imaging sequence. Fortunately, for a bandwidth this small, this can be easily accomplished using a selective pulse of the appropriate phase. Following this pulse, the flyback sequence with a small tip angle can be implemented in the standard manner.

### Example 5

### Transfer of Hyperpolarized ¹³C Technology to a Clinical 3 T Scanner

To facilitate the development of the three key innovations, a list of enabling pulse sequence features can be first programmed and tested. To begin with, while some of the preparation phases (e.g., shimming) will be utilizing the proton channel, power optimization and center frequency determination will need to tap into the non-renewable carbon polarization. Therefore, the conventional preparation phases may generally be avoided and small flip-angle versions can be used, along with options for user-initiated skipping of selected preparation phases altogether in cases where these adjustments can be performed on phantoms before the actual animal scanning.

A small double tuned (¹³C, ¹H) surface loop with diameter of 3 cm can be constructed. This can be used for initial phantom tests and experiments in living mice. Additionally, a 4-channel receive, single-channel transmit assembly resonating at the ¹³C frequency can be built for testing accelerated acquisition algorithms on hyperpolarized phantoms and in injected rats. The 4-channel receiver can be composed of two sets of 3-cm diameter surface loops, each loop interfaced to an individual preamplifier. The loops in a set can be decoupled via partial overlapping and the two sets can be isolated via the low impedance preamplifiers. A large, 10×10 cm², surface loop can be used for signal excitation on the carbon frequency. Standard cross-diodes and DC bias from the scanner can be used to isolate transmit and receive portions of the RF circuitry.

The following methods can be used to transfer the high-field solutions to a clinical 3 T system.

Acquisition-Weighted Double Spin-Echo EPSI for Rapid Hyperpolarized ¹³C CSI.

Efficient use of the available hyperpolarization requires only a small fraction of the spins to be excited for each phase encoding. One way to achieve this is to use small flip angle excitation combined with the acquisition of multiple phase encodings via EPSI. A simple spin-echo based EPSI, while insensitive to field inhomogeneities, will not suffice since the accuracy of the 180° refocusing pulse will be vulnerable to transmitter miscalibrations and the consequent loss of signal. A double-spin echo EPSI (Cunningham et al., 2007), can improve the stability of sequence to incorrect setting of the transmit gain. In addition, adiabatic refocusing pulses will be used to form the echo. Given a typical low-resolution matrix of 16×16 for hyperpolarized CSI, a significant signal contamination between adjacent voxels may be expected. An acquisition filtering of the k-space can be implemented via a variable flip angle scheme that emphasizes the center of k-space (Pohmann and von Kienlin, 2001). This can significantly reduce the sidelobes of the spatial response function and thus the contamination from adjacent voxels. The performance of this sequence can be compared to conventional CSI on a set of thermally-polarized phantoms.

Translating the high-resolution scanner sequences to a clinical 3 T system can gain the advantage of the larger BW achievable by EPSI at this lower field. This can be important if spectral aliasing is to be avoided in simultaneous acquisition of lactate, alanine, pyruvate, and bicarbonate signals that require coverage of the 161-185 ppm range. In an implementation of a single spin echo EPSI (FIG. 8), 1.6 kHz (50 ppm) BW can be achieved with spatial resolution of 5 mm. While this resolution is not sufficient for small animal cardiac imaging, some of the available spectral BW can be traded for higher resolution (since the higher gradients will require longer ramp up time and thus lower EPSI spectral BW).

FLOPSY-EPSI for Transferring Polarization from Glutamate C5 to C4 and C3.

A significant impediment in the development of hyperpolarized ¹³C tracers for metabolic imaging is the short relaxation times of protonated carbons. When hyperpolarized, these carbons can lose their enhanced signal relatively fast and before sufficient intermediary metabolites have been accumulated. Deuteration of these carbons does lengthen their relaxation times but this requires expensive ¹³C and ²H enriched compounds. Alternatively, one can use the long relaxation times of carbons that have no protons bound to them to store hyperpolarization. This enhancement can then be transferred to other carbons via homonuclear cross-polarization mixing techniques as described above. Practical considerations generally require that the cross-polarization is broadband enough to cover the large ¹³C chemical shift range: for glutamate this translates in coverage between 182 ppm (C5-glu) and 27 ppm (C3-glu). While several classic mixing techniques like WALTZ-16 and MLEV-17 can provide this they generally require prohibitively large RF fields. FLOPSY-8 will be implemented for longitudinal mixing. The critical improvement that FLOPSY-8 offers is that it can efficiently transfer polarization even when the RF mixing field strength is smaller than the chemical shift difference of the long T₁ source and the receiving spins. This is especially important because of the large chemical shift difference, 4.68 kHz, between C5 and C4 of glutamate at 3 T. Preliminary estimation of the RF field strength required to cover this bandwidth is 58.3 μT, which is easily achievable for ¹³C pulses on the 3 T scanner. Given a typical 90° ¹³C flip angle of less than 50 μs, it is expected to be able to fit the entire nine pulses (total flip angle 1060°) of a single R-unit into about 1.25 ms. Thus, the entire FLOPSY-8 can take approximately as long as τ=½J₅₄=9.6 ms, the time required for optimum polarization transfer between C5 and C4 of glutamate. This condition will be slightly relaxed for J₃₄=34 Hz for the C4 and C3 atoms. Polarization between C5 and C4, and C4 and C3 will be transferred in one run, where the length of the mixing FLOPSY-8 sequence will be optimized experimentally.

Low-SAR Adiabatic Decoupling with WURST RF Pulses.

Proton decoupling can be used to simplify the spectroscopic patterns and to increase SNR so that proper fitting of the enrichment multiplets can be performed. State of the art broadband decoupling schemes are available on many of the high resolution platforms but they often rely on the use of high power that can be prohibitive for in vivo work. In addition, the non-uniformity of the excitation fields on whole body scanners requires higher insensitivity of the decoupling to resonance offsets. A low-power adiabatic broadband decoupling scheme on the 3 T system may be used. One can decouple the full ˜6 ppm proton spectra with the limitation that the whole body coil can sustain 13.5 uT B₂max. In addition, decoupling sidebands generally need to be below 1% to avoid significant distortion in the spectral patterns. The work of Kupce and Freeman (1995a and 1995b) offers a candidate for this decoupling scheme. Their investigation of stretched hyperbolic secant RF pulses, better known as WURST pulses, show an order of magnitude higher figure of merit (defined as the ratio of the achievable decoupling BW vs. B₁ᵣₘₛ) of the WURST scheme as compared to WALTZ-16. This can be important given the limited B₁max of the integrated body coil and it can allow for trading some of the extremely wide decoupling bandwidth of WURST for better sideband suppression. WURST profiles are easily generated and will be used in a nested combination of MLEV and Tycko's 20-step phase cycling scheme (Kupce and Freeman, 1995b). The WURST pulses generated in this objective will also be used for the adiabatic dual spin-echo refocusing in EPSI.

SENSE-EPSI, Keyhole-EPSI, and Compressed-EPSI.

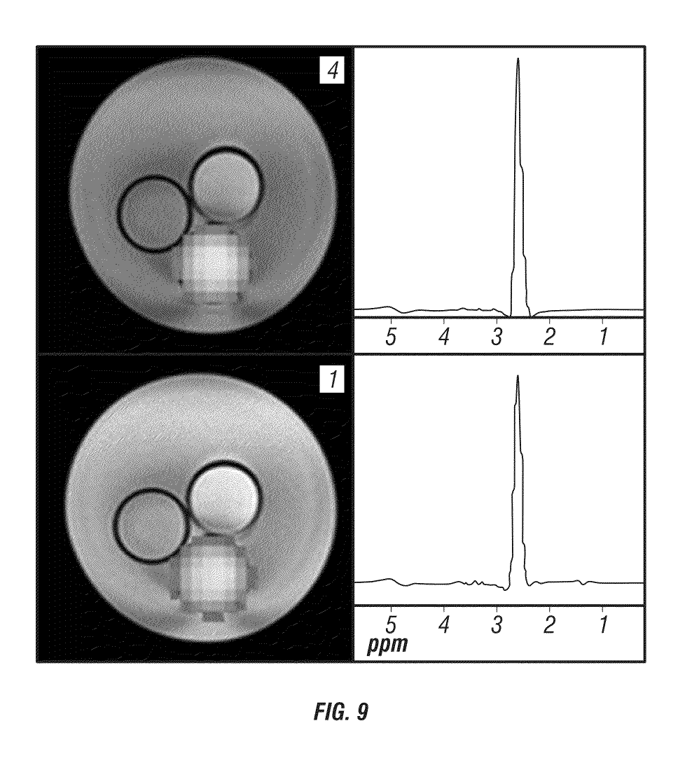

Fast acquisition of the hyperpolarized data can be useful for detecting dynamic metabolic processes in vivo. Fast acquisition of hyperpolarized data without sacrificing spatial resolution, can involve the acquisition of fewer phase encodings followed by the application of advanced mathematical algorithms to reconstruct the data. Data in FIG. 9 shows comparable spectral quality of CSI spectra acquired with conventional phase acquisition, as compared to twice-accelerated SENSE-CSI (Dydak et al., 2001). Utilizing the 4-channel ¹³C acquisition configuration accelerations up to a factor of two can be tested in both encoding directions. It is anticipated that scan times can thus be reduced by a factor of four. In some instances, no acceleration will be performed in the spectral domain. For the ¹³C SENSE-CSI (Arunachalam et al., 2009), the central portion of k-space will be fully sampled, at the pyruvate frequency, to generate the low-resolution coil sensitivity maps for the SENSE reconstruction. Studies of the expected SNR and spectral-spatial reproducibility as a function of various acceleration factors will be conducted on a set of phantoms and in small animals.

Further reduction of the number of phase encodes can be tested through the use of keyhole sampling (van Vaals et al., 1993). A fully-covered k-space mask can be generated at the peak of the expected signal function and this mask will be used to reconstruct centrally-sampled k-space maps of the rest of the dynamics. Studies have shown that only 25% of the central k-space needs to be sampled for faithful reconstruction of the full data sets. While this approach may require physiological triggering when doing myocardial CSI, it is anticipated that the additional speed achieved can allow for acquisition of the data in a limited number of triggers. Reduction of the size of k-space has been successfully used with hyperpolarized carbon (Golman et al., 208).

As a third component of this Example for more efficient hyperpolarized imaging, the novel area of compressed sensing can be tested. Accurate reconstruction of sparse spectroscopic data can be achieved by acquiring data, in kₓ and k_{y}, at sub-Nyquist sampling rates (Hu et al., 2008).

Finally, combinations of some of these methods can be examined to determine the performance of the algorithms to such experimental factors as SNR and unequal weighting of k-space due to decay of polarization.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
PCT Appln. WO-A-01/96895
PCT Appln. WO-A-02/36005
PCT Appln. WO-A-02/37132
PCT Appln. WO-A-98/30918
PCT Appln. WO-A-98/58272
PCT Appln. WO-A-99/24080
PCT Appln. WO-A-99/35508
Arunachalam et al., NMR Biomed., 22(8):867-873, 2009.
Badar-Goffer et al., Biochem, J., 266:133, 1990.
Bennett et al., J. Magn. Reson., 165:59-79, 2003.
Chen et al., Magn. Reson. Med., 58(6):1099-1106, 2007.
Cunningham et al., J. Magn. Reson., 187(2):357-362, 2007.
Dydak et al., Magn. Reson. Med., 46(4):713-722, 2001.
Golman et al., Magn. Reson. Med., 59(5):1005-1013, 2008.
Hogben et al., J. Magnetic Reson., 208:179-194, 2011.
Hu et al., J. Magn. Reson., 192(2):258-264, 2008.
Kadkhodie et al., J. Magn. Reson., 91:437-443, 1990.
Kadkhodie et al., J. Magn. Reson., 91:437-443, 1990.
Kupce and Freeman, J. Magn. Reson., 115:273-276, 1995a.
Kupce and Freeman, J. Magn. Reson., 117:246-256, 1995b.
Levin et al., Magn. Reson. Med., 58(2):245-252, 2007.
Levitt et al., J. Magnetic Reson., 47:328-330, 1981.
Malloy et al., Am. J. Physiol., 259(3 Pt 2):H987-95, 1990b.
Malloy et al., Biochemistry, 29(29):6756-6761, 1990a.
Mohebbi and Shaka, Chem. Physics Lett., 178(4):374-378, 1991.
Pohmann and von Kienlin, Magn. Reson. Med., 45(5):817-826, 2001.
Rucker and Shaka, Mol. Phys., 68(2):509-517, 1989.
Schroeder et al., FASEB J., 23:2529-2538, 2009.
Shaka et al., J. Magn. Reson., 53(2):313-340, 1983.
Sherry et al., Metab. Eng., 6:12-24, 2004.
van Vaals, J. Magn. Reson. Imaging, 1993; 3(4):671-675, 1993.
Yen et al., Magn. Reson. Med., 62(1):1-10, 2009.
Zeng et al., J. Magn. Reson., 199(2):159-165, 2009.
Zhao et al., J. Magn. Reson., 113(2):179-83, 1996.

What is claimed is:

1. A method for transfer of hyperpolarization, comprising:
obtaining a living subject, tissue or organ comprising a hyperpolarized target comprising isotopes with a nonzero nuclear spin at a first and second position, wherein the first position has a longitudinal relaxation time (T₁) longer than that that of the second position and has a hyperpolarized isotope, and both of the isotopes are not ¹H; and
transferring the hyperpolarization from the first position to the second position.

2. The method of claim 1, wherein the isotope at the first or second position is ¹³C, ¹⁹F, ³¹P, ³³S, ¹⁵N, ⁸⁹Y, ⁷⁵As, ⁶³Cu, ⁶⁵Cu, ²³Na, ⁹⁹Ru, ¹⁰¹Ru, ⁶Li, ⁷Li, ¹³³Cs, or ¹⁷O.

3. The method of claim 2, wherein the isotope is ¹³C or ¹⁵N.

4. The method of claim 1, wherein the T₁ at the first position has a value of at least 5 seconds.

5. The method of claim 1, wherein the T₁ at the second position has a value of at most 2 seconds.

6. The method of claim 1, wherein the hyperpolarized target is an intermediate in an in vivo biological process.

7. The method of claim 6, wherein the biological process is anaplerosis, glucose metabolism, ornithine cycle, GABA (γ-Aminobutyric acid) cycle, beta oxidation, tricarboxylic (TCA) cycle or urea cycle.

8. The method of claim 6, wherein the hyperpolarized target is hyperpolarized glutamate.

9. The method of claim 1, wherein obtaining the subject, tissue or organ comprises introducing a hyperpolarized precursor into the subject, tissue or organ, wherein the hyperpolarized precursor provides the hyperpolarized target in the subject, tissue or organ.

10. The method of claim 9, wherein the hyperpolarized precursor is hyperpolarized by dynamic nuclear polarization (DNP), para-hydrogen induced polarization (PHIP), or brute force polarization.

11. The method of claim 9, wherein the hyperpolarized precursor is a hyperpolarized tricarboxylic acid (TCA) cycle metabolite precursor.

12. The method of claim 11, wherein the hyperpolarized precursor is a hyperpolarized form of [U-¹³C]pyruvate, [2,3-¹³C]pyruvate, [1,2-¹³C]acetate, [U-¹³C]lactate, [U-¹³C]alanine, [1,2-¹³C]acetyl-CoA, [U-¹³C]butyrate, [1,2-¹³C]butyrate, or any compound that could produce [1,2-¹³C]acetyl-CoA.

13. The method of claim 1, further comprising administering to the subject, tissue or organ a precursor labeled with an isotope of the same type as that of the isotope at the second position of the hyperpolarized target.

14. The method of claim 13, wherein the labeled precursor is [U-¹³C]pyruvate, [U-¹³C]acetate, [U-¹³C]propionate, [1-¹³C]pyruvate, [3-¹³C]pyruvate, [U-¹³C]lactate, [U-¹³C]alanine, [U-¹³C]dihydroxyacetone, [1-¹³C]lactate, [1-¹³C]alanine, [3-¹³C]lactate, or [3-¹³C]alanine.

15. The method of claim 1, wherein the polarization transfer is homonuclear transfer.

16. The method of claim 15, wherein the homonuclear transfer is C—C transfer.

17. The method of claim 1, wherein polarization transfer is heteronuclear transfer.

18. The method of claim 1, wherein the polarization transfer comprises a J-coupling scheme.

19. The method of claim 18, wherein the J-coupling scheme comprises the use of FLOPSY (Flip-flop spectroscopy), MLEV (Malcolm Levitt's composite-pulse decoupling sequence), DIPSI (Decoupling in the presence of scalar interactions), WALTZ, or HOHAHA (Homonuclear Hartmann-Hahn).

20. The method of claim 1, wherein the polarization transfer comprises a J-modulated scheme.

21. The method of claim 20, wherein the J-modulated scheme comprises the use of INADEQUATE (Incredible natural abundance double quantum transfer) or DOUBTFUL (Double quantum transitions for finding unresolved lines), or any selective pulse versions of these transfer schemes.

22. The method of claim 1, further comprising transferring the hyperpolarization from the first position to an intermediate position before transferring to the second position.

23. The method of claim 1, further comprising detecting magnetic resonance signals in the target after the hyperpolarization transfer.

24. The method of claim 23, further comprising measuring a biochemical process in the subject, tissue or organ based on the magnetic resonance signals, wherein the biological process is selected from the group of anaplerosis, glucose metabolism, ornithine cycle, GABA (γ-Aminobutyric acid) cycle, beta oxidation, urea cycle, or TCA cycle.

25. The method of claim 24, further comprising providing a prognosis or diagnosis of the subject based on the measurement as compared to a control.

26. The method of claim 1, wherein the subject has or is at risk of having a tumor, an inflammation, an infection, a metabolic disease, a neurological disease, a cardiac disease, a liver disease, a kidney disease, or diabetes.

27. The method of claim 1, wherein the organ is heart, brain, liver, or kidney.

28. The method of claim 1, wherein the organ is ischemic or malignant.
